(19) United States
(12) Patent Application Publication
(10) Pub. No.: US 2002/0071095 A1
(43) Pub. Date: Jun. 13, 2002

(54) Composite surface contact lenses

(76) Inventors: Jefrey H. Roffman, Jacksonville, FL (US); Michael J. Collins, Mt. Nebo (AU); Gregory Hofmann, Jacksonville, FL (US); Brett A. Davis, Cooparoo (AU)

Correspondence Address: Philip S. Johnson, One Johnson & Johnson Plaza, New Brunswick, NJ, 08933-7003, US

(21) Appl. No.: 09/732,646

(22) Filed: Dec. 8, 2000

(51) Int. Cl. A61B 3/00

(52) U.S. Cl. 351/161; 351/247; 351/219

(57) ABSTRACT

The present provides lenses in which the one surface incorporates wavefront aberration correction as well as corneal topographic data. Additionally, the invention provides methods for manufacturing such lenses.

### FIELD OF THE INVENTION

The present invention relates to the design and manufacture of ophthalmic lenses. In particular, the invention provides lenses one surface incorporates both wavefront aberration correction as well as corneal topographic data.

### BACKGROUND OF THE INVENTION

The use of contact lenses for the correction of ametropia is well known. A limitation of conventional contact lenses is that the lenses correct only for an individual's basic, sphero-cylindrical ametropia, or low order aberrations, leaving higher order aberrations of the eye uncorrected. Additionally, conventional contact lenses do not take into account aberrations due to corneal topography. Recently, certain lenses have been developed that provide correction for high order aberrations on one or both surfaces. Additionally, lenses have been developed in which one or more surfaces provides correction for aberrations due to corneal topography. However, a need exists for a lens that combines correction for both higher order aberrations and aberrations due to corneal topography on a single surface.

### DESCRIPTION OF THE INVENTION AND ITS PREFERRED EMBODIMENTS

The present invention provides methods for designing contact lenses and lenses produced by those methods. The lenses of the invention provide correction for low and high order aberrations as well as aberrations due to corneal topography. In particular, one surface of the lens provides correction for high order wavefront aberrations and aberrations due to corneal topography.

Thus, in one embodiment, the invention provides a contact lens comprising, consisting essentially of, and consisting of a surface that corrects high order ocular aberrations and aberrations due to corneal topography. In another embodiment, the invention provides a method for designing a contact lens comprising, consisting essentially of, and consisting of the steps of: a.) obtaining corneal topographic data for an eye of an individual; b.) measuring high order ocular aberrations for the eye of the individual; and c.) providing a surface for the contact lens that corrects the high order ocular aberrations and aberrations due to the corneal topography.

For purposes of the invention, by “low order ocular aberration” is meant an aberration that causes basic, sphero-cylindrical ametropia in an individual. Such aberrations are typically corrected using sphere and cylinder powers. By “high order ocular aberration” is meant an aberration, other than low order aberrations and aberrations due to corneal topography, that results from the difference between the wavefront emerging from the eye and a perfect wavefront.

Corneal topographic data, or information, for an individual's may be obtained using any of a number of known devices. Generally, the data is obtained using a corneal topographer or videokeratoscope. Preferably, a topographer with high resolution along the z-axis is used. The data is acquired above and below the mean spherical surface of the cornea parallel to the longitudinal axis of the cornea. The data may be of the anterior cornea surface, the posterior corneal surface, or both.

Once obtained, the corneal topographic data is mathematically transformed to a form suitable for use in the design and production of a lens. For example, the topographic data may be used to determine the elevation map of the lens' back, or eye side surface, front surface, or object side surface, or a combination thereof by mapping of the corneal elevation onto a lens surface by any known method. For soft contact lens production, preferably, mapping is carried out so that the error introduced by flexure of the lens is minimized.

For lenses in which the data is incorporated into the front surface, preferably the corneal elevation data is applied to a soft contact lens in the unflexed state and then the elevation data is transformed by taking into account lens flexure. In such a method, for practical considerations, it may be assumed that the ideal cornea is spherical and that the actual corneal elevations and their best spherical fit are denoted f(x) and g(x), the function g(x) being part of a sphere having radius Rₐ. In general, the radius R_{b} of an unflexed soft contact lens is spherical and is larger than that of the best spherical fit g(x). The first step is to transform the corneal elevations f(x) into a larger scale for which the best spherical fit will have a radius equal to R_{b}. One approach in simplifying the transformation is to represent the function f(x) in polar coordinates as f(θ). Then using the scale factor α=R_{b}/Rₐ, the scaled version of the corneal elevation may be expressed as:

f⁽¹⁾(θ)=αf(θ)

In the second stage, the scaled corneal elevation, f (θ) is scaled down so that the area covered by the soft contact lens corresponds to the area of the cornea. In a two dimensional case, this scaling down is obtained according to the following relationship:

f⁽²⁾(θ)=α⁻¹f⁽¹⁾[(θ)−π/2)/α+π/2]+R_{b}(1−1/α)

The mapping transformations given in the above equations are not restricted to the case in which the cornea and the back surface of the contact lens are spherical. Rather, the true corneal and lens curvatures may be used to calculate the scale parameter α: as a ratio between the lens and the corneal radius of curvature. In the general case, the scale parameter will be a function of θ, i.e., α=R_{b}(θ)/Rₐ(θ)=α(θ).

The mapping transformation discussed above may be generalized to the case of three dimensional transformation. In such a case, the corneal elevations may be represented by a function, f(θ,φ) where θ and φ represent the azimuth and elevation angle, respectively. The original elevation data is scaled up from the radius of curvature Rₐ (θ,φ) using the following transformation relationship:

f⁽¹⁾(θ,φ)=αf(θ,φ)

where α=R_{b}(θ,φ)/Rₐ(θ,φ).

To obtain a desired surface of the lens, the functional f⁽¹⁾(θ,φ)is scaled back down. However, in the three dimensional case, there are a number of options to choose from in performing the scaling operation such that the area is preserved. For example, if it is assumed that the deformation of the material is uniformly radial, the scaling mat be performed by scaling the elevation angle only, leaving the original azimuth angle. This is expressed in the following relationship:

f⁽²⁾(θ,φ)=α⁻¹f⁽¹⁾[(θ,(φ−π/2)/α+π/2]+R_{b}(1−1/α)

In addition to providing correction for aberration due to corneal topography on a surface, correction for high order ocular aberrations are provided on the same surface. Ocular wavefront aberrations of the eye, such as high order aberrations, are measured using any suitable device for performing aberration measurement. Suitable devices include, without limitation, aberroscopes, devices that measure ocular Modulation Transfer Function by point spread or line spread, or any similar devices that measure, estimate, interpolate, or calculate the ocular optical wavefront.

Once measured, the aberration measurements are mathematically converted to a height difference, thus providing an elevation map above and below a designated mean sphere value, known as the optical path difference. For example, the elevation map may be created by multiplying the wavefront error, as measured in optical waves, by the wave length, point-by-point, across the wavefront. Correction for the aberrations will be provided by introduction of an optical path difference, or aberration inverse filter, that offsets the distortions due to the ocular aberrations. In the lenses of the invention, this correction is provided on the same surface into which the corneal topographic data is incorporated, which surface is preferably, the front surface of the lens.

In addition to high order aberration measurement, low order aberrations may be measured to provide the cylinder power and axis along with the sphere power for correction of distance vision, and, optionally, the near and intermediate vision acuity. These measurements may be carried out by any method including by the use of conventional refractive techniques. Alternatively, and preferably, these measurements may be determined via ocular wavefront aberration measurement. For example, this may be carried out by reducing wavefront data to Zernike coefficient terms and using this information to derive the sphere, cylinder, and axis information.

Using the topographic data and high order aberration measurements, a surface of a lens is designed. Any number of embodiments of the lens of the invention are possible. In one embodiment, the topographic data for a cornea is measured using a corneal topographer and high order ocular aberrations are measured. The back surface of a lens then is designed to neutralize aberrations due to the corneal topography and high order ocular aberrations. The optic zone of both surfaces of the lens has the sphere power, cylinder power, or both necessary for correction of low order aberrations.

In a preferred embodiment, corneal topographic data is obtained and used to estimate the print-though of an individual's corneal topography from the back surface of the lens to the front surface. Alternatively, a conventional lens having substantially the corrective power necessary to correct the low order aberrations may be placed onto the individual's eye and the actual print through may be measured. The front surface is designed so as to neutralize any aberrations due to this print through. The high order aberrations are then determined and the front surface of a lens is designed to neutralize aberrations due to the corneal topography and high order ocular aberrations. Alternatively, the net residual aberrations may be determined by measuring total ocular wavefront aberrations and by subtracting those due to the corneal print through from the total aberrations measured. This net residual aberration, which includes both high and low order aberrations, then may be compensated for by an appropriate design of the front surface.

In a more preferred embodiment of the lens of the invention, correction for high order ocular aberrations and aberrations due to corneal topography are provided on a single surface and decentering relative to the lens wearer's line of sight is used. More specifically, the topographic map is decentered from the mechanical center of the lens to the vertex normal and the wavefront aberration is centered on the line of sight. Preferably, the decentering is about 0 to about 1.5 mm.

The decentering may be carried out by any convenient method. For example, the vertex normal in a corneal topography map is the point on the cornea at which the slope is perpendicular to the axis of a videokeratoscope cone. Thus, the central videokeratoscope ring is reflected straight back to the camera. For purposes of aligning a lens with the surface topography, the location of the vertex normal relative to the edge of the limbus is required. Corneal topography is measured in any convenient manner and the map to be used is selected. A transparent geometric center overlay template is used to find the geometric center position of the topographic map with respect to the pupil. The template may be of any suitable design. A conveniently used template has concentric rings extending outwardly from its center. Such a template may be positioned so that the rings are concentric with the eye's limbus.

The lenses of the invention may be produced using any known method. Suitable methods include, without limitation, lathing or molding the lenses. For example, the lens design may be cut into a metal and the metal used to produce plastic mold inserts for the lens' surfaces. A suitable liquid resin is then placed between the inserts, the inserts compressed, and the resin cured to form the lens. Alternatively, the lens of the invention may be produced by cutting the lens on a lathe.

The lenses of the invention may be made from any suitable materials for manufacturing hard or soft contact lenses. Preferably, the lenses are soft contact lenses. Illustrative materials for formation of soft contact lenses include, without limitation silicone elastomers, silicone-containing macromers including, without limitation, those disclosed in U.S. Pat. Nos. 5,371,147, 5,314,960, and 5,057,578 incorporated in their entireties herein by reference, hydrogels, silicone-containing hydrogels, and the like and combinations thereof.

In yet another embodiment, the invention provides a method for manufacturing contact lenses comprising, consisting essentially of and consisting of the steps of: a.) obtaining data for an individual comprising one or more of corneal topographic data, low order ocular aberrations, and high order ocular aberrations; b.) transmitting to a manufacturer the data obtained in step a.); c.) generating a lens design using the data; and d.) manufacturing a lens based on the lens design. Step a.) may be carried out by any suitable entity, including, without limitation, optometrists, opticians, lens retailers, and the like. Preferably, the method of the invention is carried out so that it is a business-to-business system.

Generation of the lens design typically will be carried out by the lens manufacturer. The data may be transmitted to the manufacturer by any suitable method including, without limitation, telephone, facsimile transmission, internet website, and the like and combinations thereof. In a preferred embodiment, transmission is carried out via the lens manufacturer's internet website by the customer using any means capable of communicating with the lens manufacturer's server system (web server or web site). Suitable means for communicating with the website include, without limitation, a personal computer and modem. Preferably, a data file is created that may be uploaded to the manufacturer's web server database.

What is claimed is:

1. A contact lens comprising a first surface that corrects high order ocular aberrations of the eye and aberrations due to corneal topography of the eye.

2. The lens of claim 1, wherein the first surface is a front surface of the lens.

3. The lens of claim 1, wherein the first surface is a back surface of the lens.

4. The lens of claim 1, wherein the correction for the aberrations due to corneal topography are decentered relative to a vertex normal.

5. A method for designing a contact lens comprising the steps of a.) obtaining corneal topographic data for an eye of an individual; b.) measuring high order ocular aberrations for the eye of the individual; and c.) providing a surface for the contact lens that corrects the high order aberrations and aberrations due to the corneal topography.

6. The method of claim 5, further comprising measuring the low order ocular aberrations of the eye and providing sphere power, cylinder power, or both for correction of the low order ocular aberrations.

7. The method of claim 6, wherein the surface provided for correcting for the high order ocular aberrations and aberrations due to corneal topography is a back surface of the lens.

8. The method of claim 6, wherein the surface provided for correcting for the high order ocular aberrations and aberrations due to corneal topography is a front surface of the lens.

9. The method of claim of claim 5, further comprising the step of decentering the correction for the aberrations due to corneal topography relative to a vertex normal.

10. The method of claim of claim 6, further comprising the step of decentering the correction for the aberrations due to corneal topography relative to a vertex normal.

11. The method of claim of claim 7, further comprising the step of decentering the correction for the aberrations due to corneal topography relative to a vertex normal.

12. The method of claim of claim 8, further comprising the step of decentering the correction for the aberrations due to corneal topography relative to a vertex normal.

13. A method for manufacturing contact lenses comprising the steps of: a.) obtaining data for an individual comprising one or more of corneal topographic data, low order ocular aberrations, and high order ocular aberrations; b.) transmitting to a manufacturer the data obtained in step a.); c.) generating a lens design using the data wherein the lens provides correction for one or more of the low order ocular aberrations, high order ocular aberrations, or aberrations due to the corneal topography; and d.) manufacturing a lens based on the lens design.

14. The method of claim 13, wherein step b.) is carried out by creating a data file that may be uploaded to a web server database of the manufacturer.

15. The method of claim 13, further comprising the step of providing correction for the high order ocular aberrations and the aberrations due to the corneal topography on one surface of the lens.

16. The method of claim 13, further comprising the step of decentering the correction for the aberrations due to corneal topography relative to a vertex normal.

17. The method of claim 15, further comprising the step of decentering the correction for the aberrations due to corneal topography relative to a vertex normal.

18. A lens produced by the method of claim 5.

19. A lens produced by the method of claim 15.

20. A lens produced by the method of claim 17.
